(19) United States
(12) Patent Application Publication
(10) Pub. No.: US 2024/0174964 A1
(43) Pub. Date: May 30, 2024

(54) CELL DETACHMENT DEVICE, VIBRATION ELEMENT, CELL DETACHMENT DEVICE CONTROL METHOD, AND MEDIUM

(71) Applicant: CANON KABUSHIKI KAISHA, Tokyo (JP)

(72) Inventors: TAKUJI OKAMOTO, Tokyo (JP); TAKAYUKI TSUKIMOTO, Kanagawa (JP); NORIHIRO SUZUKI, Kanagawa (JP)

(21) Appl. No.: 18/509,495

(22) Filed: Nov. 15, 2023

(30) Foreign Application Priority Data
Nov. 30, 2022 (JP) 2022-192053

(51) Int. Cl. C12M 1/26; H10N 30/00; H10N 39/00

(52) U.S. Cl. CPC C12M 33/08; H10N 30/1071; H10N 39/00

(57) ABSTRACT

Provided is a cell detachment device causing to know whether the vibration applied to a culture vessel and the cells under the desired condition is applied. The device generating a first vibration and detaching a culture cell from a culture vessel using the first vibration, comprises: a piezoelectric element in which a second vibration is excited by applying a first alternating current using a first and second electrodes; a vibrated system in which the second vibration propagates, including a vibration member to which the piezoelectric element is fixed and on which the culture vessel mounted; a third electrode used outputting a second alternating current excited on the piezoelectric element by the first vibration generated in the vibrated system caused by the second vibration; and a detection unit detecting a state of the first vibration generated in the vibrated system by using the second alternating current output from the third electrode.

### BACKGROUND OF THE INVENTION

### Field of the Invention

The present disclosure relates to a cell detachment device, a vibration element, a control method for a cell detachment device, and a medium.

### Description of the Related Art

In the medical field, cells and the like may be cultured in culture vessels such as culture plates and dishes (petri dishes) for treatment or research and development. However, the cultured cells often adhere a bottom surface of the culture vessel, and in such cases, it is necessary to detach the cells so as to remove the cells from the culture vessel.

Methods for such detachment of the cells includes a method of applying enzymes or chemicals acting on the cell membrane to detach the cells, a method of using a temperature-responsive polymer to detach the cells, or a method of applying vibration energy to the cells by injecting ultrasonic waves to detach the cells. A document of Japanese Patent Application Laid-Open No. 2011-115080 discloses a cell detachment device in which an ultrasonic wave emitting means is disposed apart from the outer surface of the culture vessel, and the ultrasonic wave emitting means and a region to be processed on the outer surface of the culture vessel are connected by an ultrasonic transmitter. In the cell detachment device in the document of Japanese Patent Application Laid-Open No. 2011-115080, a distance sensor is disposed below the culture vessel, and the distance between the sheet-like cells separated from the bottom surface of the vessel by the ultrasonic vibration and the bottom surface of the vessel is measured, and whether the cells are detached from the culture vessel is determined using the measurement result.

### SUMMARY OF THE INVENTION

When vibration such as ultrasonic wave is applied to the culture vessel to induce detachment of the cells, sufficient detachment may not be obtained when the vibration is small or the applying time of vibration is short. In addition, when the vibration is excessive or the applying time is too long, cell damage may be caused.

Therefore, in a cell detachment device, vibration must be continued for a predetermined time at the vibration frequency and vibration amplitude set for the cell detachment. In the cell detachment device disclosed in the document of Japanese Patent Application Laid-Open No. 2011-115080, it is possible to determine whether the cells adhere to the culture vessel. However, it is not possible to know whether the vibration applied to the culture vessel and the cells is too large or too small, and whether the cell detachment device is in a state where the vibration applied to the culture vessel and the cells according to the set condition is applied.

In view of the above circumstance, one of the purposes of an embodiment of the present disclosure is to know whether the vibration applied to the culture vessel and the cells under the desired condition is applied in the cell detachment device.

To solve the above problems, a cell detachment device according to an embodiment of the present disclosure, is a cell detachment device generating a first vibration and detaching a culture cell from a culture vessel by using the first vibration, comprising:
a piezoelectric element in which a second vibration is excited by applying a first alternating current using a first electrode and a second electrode;
a vibrated system in which the second vibration propagates, including a vibration member to which the piezoelectric element is fixed and the culture vessel mounted on the vibration member;
a third electrode used configured to output a second alternating current excited on the piezoelectric element by the first vibration generated in the vibrated system caused by the second vibration; and
a detection unit configured to detect a state of the first vibration generated in the vibrated system by using the second alternating current output from the third electrode.

Further features of the present invention will become apparent from the following description of exemplary embodiments with reference to the attached drawings.

### BRIEF DESCRIPTION OF THE DRAWINGS

FIG. 1A shows a schematic configuration of a device main body 1 of a cell detachment device.

FIG. 1B shows an example of a piezoelectric element provided with electrodes.

FIG. 1C shows an example of a vibration state of a vibration plate to which a piezoelectric element is fixed.

FIG. 2 is a block diagram showing an example of a control unit of a cell detachment device according to the first embodiment.

FIGS. 3A and 3B are diagrams showing an example of a state in which a sensor electrode is formed on a piezoelectric element.

FIGS. 4A and 4B are diagrams showing another example of a state in which a sensor electrode is formed on a piezoelectric element.

FIGS. 5A and 5B are diagrams showing another example of a state in which a sensor electrode is formed on a piezoelectric element.

FIG. 6 is a diagram for explaining a method for detecting an abnormal state when vibration is added.

FIG. 7 is a diagram for explaining a lead-out line from a sensor electrode according to Example 3.

FIG. 8 is a diagram for explaining remote communication according to Example 4.

FIG. 9 is a diagram showing a schematic configuration of an example of a vibration element used in a cell detachment device according to the second embodiment.

FIG. 10 is a diagram showing a schematic configuration of an essential part of an example of a cell detachment device according to the second embodiment.

FIG. 11A is a diagram showing an axial section of a swinging type vibration element used in a cell detachment device according to the second embodiment.

FIG. 11B is a diagram showing the relationship between the piezoelectric element and the formation arrangement of each electrode used in a cell detachment device according to the second embodiment.

FIG. 11C is a diagram showing a top view of a flexible printed circuit board from which wiring is drawn from each electrode used in a cell detachment device according to the second embodiment.

FIG. 12 is a diagram showing an example of a piezoelectric element for vibration detection according to the third embodiment.

FIG. 13 is a diagram showing another example of a piezoelectric element for vibration detection according to the third embodiment.

### DESCRIPTION OF THE EMBODIMENTS

Exemplary embodiments and examples of the present disclosure are described in detail below with reference to the accompanying drawings. However, the dimensions, materials, shapes, and relative positions of components described in the following embodiments and examples are optional and may be modified depending on the configuration or various conditions of the device to which the present disclosure applies. In addition, in the present specification and drawings, components having substantially the same or functionally similar configuration are omitted from the duplicate description by attaching the same reference numerals between the drawings. In the following, the vibration applied to the culture vessel is defined as ultrasonic vibration, but the vibration applied is not limited to those having a frequency in the ultrasonic range, and also includes the case where the so-called vibration of extremely low frequency is applied. The cells to be detached may be isolated as individual cells, a plurality of cells may be attached to each other, and the cells may be in a sheet shape (cell sheet).

### <FIRST EMBODIMENT>

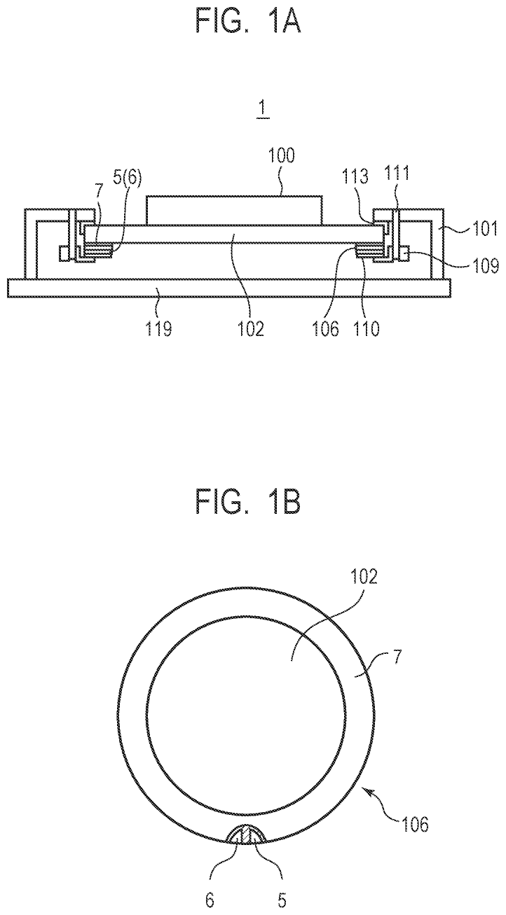

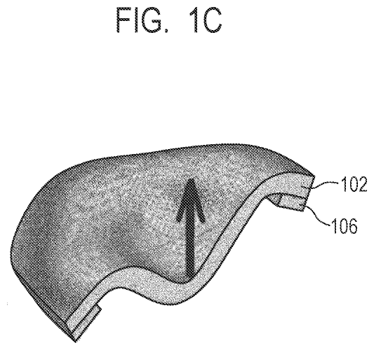

Hereinafter, as a first embodiment of the present disclosure, an example of a cell detachment device, a vibration element, and an operation method of the cell detachment device according to the present disclosure will be described with reference to FIGS. 1A to 3B.

The cell detachment device according to the present embodiment includes a device body 1, a control device 200, and a monitor 300. Hereinafter, the device body 1 and the control device 200 will be described in this order. FIG. 1A shows a cross sectional view of a schematic configuration of the device body 1 of the cell detachment device, FIG. 1B shows an example of a piezoelectric element provided with electrodes, and FIG. 1C shows an example of a vibration state of a vibration plate to which the piezoelectric element is fixed. In the following description, the vertical direction in which the culture vessel 100 is mounted on the cell detachment device is schematically determined as a direction along from upper side to lower side.

The device body 1 of the cell detachment device shown as an example of the present embodiment includes a housing 101, a piezoelectric element 106, a vibration plate 102, a support member 109, a cushion member 110, and an elastic seal member 113. The housing 101 consists of a frame-like member having a circular hole in the center of the upper surface, for example, and is placed on a work desk 119, a measuring table, or a microscope stage, where the cell detachment device is installed. Inside the circular hole, for example, the cushion member 110 is held in an upward release state through the support member 109 fixed to the housing 101 by a fixing screw 111. For the cushion member 110, a material such as felt, which has a small frictional force on an object when the object is placed on the upper surface and a small regulating force on vertical movement, is used.

The annular or ring-shaped piezoelectric element 106 is fixed to the lower surface of the flat disc-shaped vibration plate 102 composed of metal, glass, or the like. As will be described in detail later, as shown in FIG. 1B, a driving electrode 7 and a sensor electrode 5 are provided on the upper surface (the fixed surface to the vibration plate 102) of the piezoelectric element 106, and a ground electrode 6 is provided on the lower surface. The piezoelectric element 106 and the vibration plate 102 constitute the vibration element in the present embodiment. By applying an alternating current to the piezoelectric element 106 using the driving electrode 7 and the ground electrode 6, the vibration having a frequency in the ultrasonic range can be generated in the piezoelectric element 106.

The vibration element is disposed to be mounted (placed) on the cushion member 110 so as to come into contact with the cushion member 110 at the forming surface (lower surface) of the ground electrode 6 of the vibration element within the circular hole of the housing 101. Thus, by making the outer peripheral portion of the vibration element in an unrestrained state so as to make the vibration element being capable of vibrating, it is possible to generate vibration in the mode shown in FIG. 1C, for example, in the vibration element according to the present embodiment. In other words, in the vibration element according to the present embodiment, vibration can be generated using the outer peripheral portion as a free end.

An outer peripheral portion of the upper surface of the vibrating plate 102 is substantially opposed to the inner peripheral portion of the hole of the upper plate of the housing 101 provided with a circular hole, and the elastic sealing member 113 composed of rubber, for example, is disposed therebetween. In a recessed portion formed by the circular hole and the vibration plate 102 forming the bottom surface thereof, a culture vessel 100 in which the cells to be detached adhere disposed. In the cell detachment device, a vibration transmitter such as water or glycerin is put into the recessed portion. It is required that the elastic sealing member 113 does not inhibit vibration of the vibration plate 102 and seals the space between the housing 101 and the vibration plate 102 to prevent leakage of the vibration transmitter. For this reason, it is desirable to use silicone rubber or the like which has excellent chemical resistance and water resistance and relatively little vibration damping.

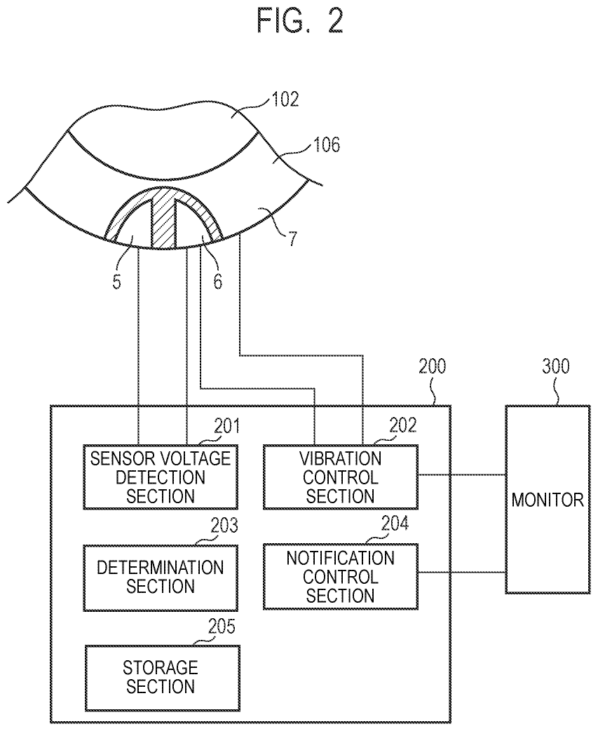

Next, the control device 200 will be described with reference to FIG. 2. The controller 200 according to the present embodiment includes a sensor voltage detection section 201, a vibration control section 202, a determination section 203, a notification control section 204, and a storage section 205. The sensor voltage detection section 201 is connected to the aforementioned sensor electrode 5 and ground electrode 6. The sensor voltage detection section 201 is enabled to detect a voltage generated in the piezoelectric element 106 by applying vibration of the vibrated system including the vibration plate 102, the culture vessel 100, the vibration transmitter and the like subjected to vibration generated by the piezoelectric element 106 to the piezoelectric element 106. The vibration control section 202 is connected to the driving electrode 7 and the ground electrode 6, and is enabled to apply the driving voltage corresponding to the vibration condition specified by the user to the piezoelectric element 106 through the input section (not shown). The vibration control section 202 may be connected to the monitor 300 including, for example, a touch panel, and the user may input the vibration condition or instruct to start or stop the vibration through the display screen of the monitor 300.

The determination section 203 performs processing such as AD conversion to the generated voltage of the piezoelectric element 106 detected by the sensor voltage detection section 201. On the basis of the obtained voltage value, it is possible to determine the appropriateness of the vibration generated in the above-described vibrated system at the time of detection. The notification control section 204 is connected to the monitor 300, and the determination result of the determination section 203 can be displayed on the monitor 300 in accordance with a predetermined format. For example, the storage section 205 can store the determination criterion of the determination section 203 based on the relationship between the input current to the driving electrode 7 and the voltage value output by the sensor electrode 5 in accordance with the input current, a program for operating the notification control section 204 in accordance with the determination result of the determination section 203, and the like. Although the control device 200 and the monitor 300 are shown as separate structures in the illustrated example, they may be integrated personal computers, and a tablet personal computer or other portable terminal may be substituted for them.

As described above, at the time of cell detachment, vibration having a predetermined amplitude must be continuously applied for a predetermined period of time. At this time, for example, it is known that a sweep of vibration frequency from 20 Hz to 30 Hz is performed, and cell detachment is most effectively performed under the condition that the vibrated system resonates within that range. However, at the time of application of vibration for the detachment, the condition of resonance may change due to a factor such as a displacement of the position of the culture vessel 100 in the vibration transmitter, and the mode of vibration of the vibrated system may change. As in the cell detachment device according to the present embodiment, by detecting the mode of vibration of the vibrated system using the sensor electrode 5, determining its appropriateness and notifying the user, it is possible to know the excessive addition of unexpected vibration to the cell and the lack of vibration.

Here, with reference to FIG. 1C, which schematically shows the state in which the vibration element including the vibration plate 102 and the piezoelectric element 106 is actually vibrating, the vibration modes generated by the vibration plate 102 and the piezoelectric element 6 will be described. FIG. 1C shows a state in which the deformation magnification is increased in the case of the vibration generation in the vibration element, the center of the perspective view is cut, and these are viewed from the cut plane side. As can be understood from the figure, a concave portion in the center of the vibration element is a portion designating the maximum amplitude, and the amplitude of the concentric striped convex part appears outside. Therefore, the outer edge of the vibration element always vibrates. It can be seen that the vibration node is formed at a position slightly inward from the outer edge of the vibration element as a part with little influence of vibration amplitude.

From the above, it is understood that the sensor electrode 5 is preferably formed at a position being partially on the outer edge of the vibration element. It should be noted that the outer edge described here means outside the center line of the cross section of the piezoelectric element 106, which is the neutral axis (approximately intermediate position between the outer and inner periphery) of the cross section. Thus, even if the voltage generated in the piezoelectric element 106 by the vibration of the vibrated system is weak, the voltage at a position with a large vibration amplitude can be measured, and the detection efficiency can be increased. The ripple-shaped vibration wave mode of the vibration element can be changed by selecting the frequency of the stretching vibration of the piezoelectric element 106. By making the outer edge of the vibration element a free edge by using an elastic member or the like, the outer edge can vibrate firmly as long as the vibration is concentric. In the present embodiment, since the shape and frequency of the vibration wave in the vibration mode are known in advance, the voltage value and frequency range of the alternating current applied to the piezoelectric element 106 are determined in accordance with this vibration condition.

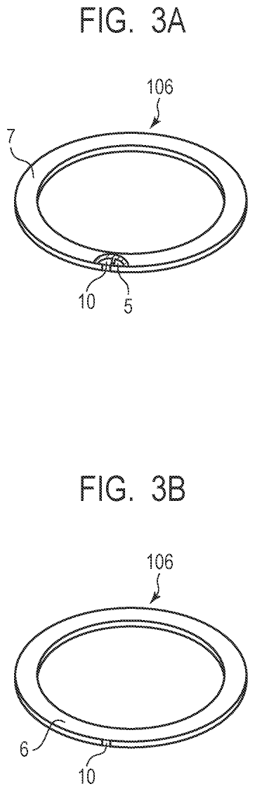

Next, the formation aspect of the sensor electrode 5, the ground electrode 6, and the driving electrode 7 for the piezoelectric element 106 will be described with reference to FIGS. 3A to 5B. FIG. 3A is a perspective view showing an upper surface (a fixed surface to the vibration plate 102) of the annular piezoelectric element 106 having a plurality of electrodes formed as exemplified in FIG. 1B, and FIG. 3B is a perspective view showing a lower surface of the piezoelectric element 106. A driving electrode 7 is formed in almost the entire upper surface of the piezoelectric element 106, and a sensor electrode 5 and a folding part 10 of the ground electrode 6 are formed in part electrically separated from the driving electrode 7. A ground electrode 6 is formed in the entire lower surface of the piezoelectric element 106, and a part of the ground electrode 6 is connected to the folding part 10 of the upper surface via an annular outer peripheral end surface.

The piezoelectric element 106 vibrates with the ground electrode 6 by applying an alternating current to the driving electrode 7, and transmits the vibration to the vibration transmitter and the culture vessel 100 through the fixed vibration plate 102. The vibrated system consisting of the vibration plate 102, the vibration transmitter (transmitting liquid), and the culture vessel 100 vibrates by the given vibration. The piezoelectric element 106 fixed to the vibrated system is subjected to compressive and tensile forces by the vibration, thereby generating a time-varying voltage in the piezoelectric element 106. By measuring thus generated voltage between the sensor electrode 5 and the ground electrode 6, the vibration state of the vibrated system can be grasped. In the present embodiment, this voltage is detected by the sensor voltage detection section 201, and the vibration state of the vibrated system is determined based on the voltage detected by the determination section 203.

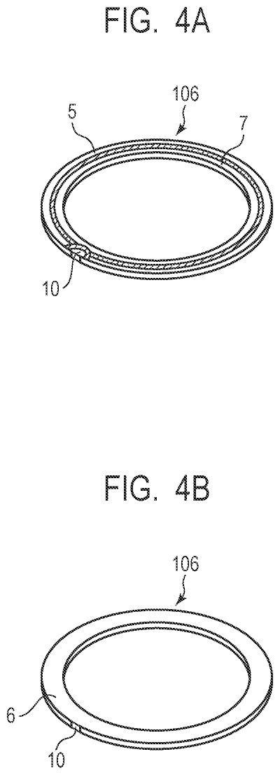

FIGS. 4A and 4B show a further formation aspect of the sensor electrode 5, the ground electrode 6, and the driving electrode 7 in the same manner as FIGS. 3A and 3B. In the illustrated example, although the ground electrode 6 is formed over the entire lower surface, the driving electrode 7 is formed on the inner peripheral side of the upper surface, and the sensor electrode 5 is formed on the outer peripheral side. In this example, the formation area of the driving electrode 7 decreases, thereby reducing the efficiency of vibration generation, but the formation area of the sensor electrode 5 increases, thereby effectively detecting the weak voltage generated by the vibration of the vibrated system.

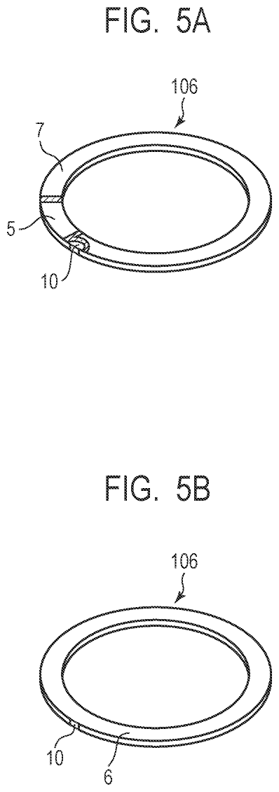

FIGS. 5A and 5B show further formation aspects of the sensor electrode 5, the ground electrode 6, and the driving electrode 7 in the same manner as FIGS. 3A and 3B. In the illustrated example, a portion of the upper surface of the piezoelectric element 106 is used for the sensor electrode 5, and the drive electrode 7 does not form an annular shape. By adopting such a formation aspect, the upper surface of the piezoelectric element 106 is partially divided into a complete region of the driving electrode 7 and a complete region of the sensor electrode 5, and a region for generating vibration and a region for detecting vibration are separated. By adjusting the arrangement of the electrodes and the formation ratio of the drive electrode 7 and the sensor electrode 5, it is possible to adjust an appropriate condition for generating vibration and the detection efficiency of vibration of the vibrated system. In the above example, the driving electrode 7 is provided on the vibration plate 102 side, but the ground electrode 6 may be provided on the vibration plate 102 side, and the driving electrode 7 and the sensor electrode 5 may be provided on the opposite side.

### (EXAMPLE 1)

Next, a method of determining an abnormality of vibration in the vibrated system and a method of notifying the result of the determination using the cell peeling detachment device according to the above embodiment will be described as an example. In Example 1 of the present disclosure, when an abnormality is detected, for example, in the sensor electrode 5, the monitor 300 displays the fact.

Specifically, an LED light or the like is provided in the monitor 300, or when there is no monitor 300 to which the control device 200 is connected, for example, an LED light or the like is provided in the control device 200, and the color of the lighting, the lighting period, and the like are controlled by the notification control section 204. As an example, when the power supply of the cell detachment device is turned ON, the notification light turns on green, and the detachment operation can be started by, for example, pressing an operation start button (not shown) displayed on the monitor 300. In the detachment operation, the green light can be made to flash, the green light can be changed to the red light when an abnormality is detected, and the red light can be made to flash when a disconnection is expected. These controls are executed according to the display program stored in the storage section 205. In addition, the monitor 300 can appropriately display these states in a message format, for example. Here, the determination section 203 determines that the operation is normal when a predetermined range of voltage values is detected, determines that the operation is abnormal when other voltage values are detected, and determines that the line is expected to break when the voltage value is 0 V.

### (Example 2)

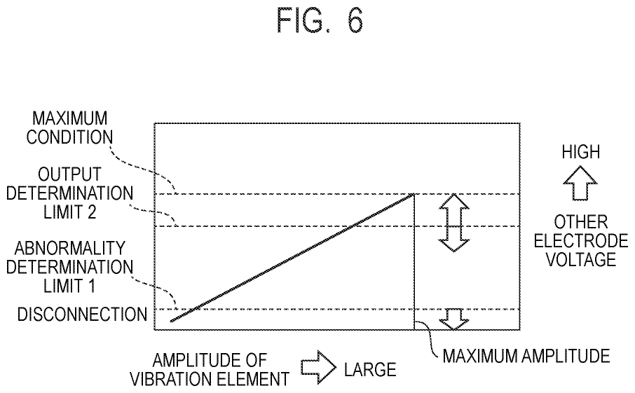

In Example 2, a case in which the determination section 203 determines the degree of deterioration of the cell detachment device by a criterion different from that of Example 1 will be described with reference to FIG. 6. In FIG. 6, the vertical axis indicates the maximum voltage value of the sensor electrode 5, and the horizontal axis indicates the amplitude in the center axis direction of the vibration plate 102 or the like shown in FIG. 1A. As shown in the figure, if the maximum amplitude equal to or greater than an output determination limit 2 designated in the figure is obtained in the region of the maximum voltage value (maximum condition), the deterioration of the cell detachment device is acceptable, and if the device is driven at a predetermined voltage set in advance, it can be determined that the operation is normal. On the other hand, if the amplitude of the vibration becomes smaller than the output determination limit 2, it indicates that the cell detachment device is deteriorated. At this time, the notification control section 204 causes the monitor 300 to display that it prompts the maintenance of the cell detachment device. At this time, the monitor 300 may display a countermeasure method corresponding to a degree of voltage drop, for example, by determining the degree of the voltage drop by the determination section 203 with an artificial intelligence. An abnormality determination limit 1 and the output determination limit 2 are stored in the storage section 205, and the countermeasure method is displayed according to a program stored in the storage unit 205.

In addition, if the detected voltage is less than the abnormality determination limit 1, the disconnection or the like is suspected, and the use of the cell detachment device is immediately stopped. In this case, the notification control section 204 may cause the monitor 300 to display the fact, and the vibration control section 202 may automatically stop the operation of the cell detachment device. Since the voltage value obtained from the sensor electrode 5 can be treated as a numerical value by, for example, converting the voltage value by a comparator, etc., the converted value is used to determine the voltage value.

Here, the sensor electrode 5 generates a voltage corresponding to the sum of the deformation of the vibration plate 102 and the strain deformation caused by the application of an alternating current to the piezoelectric element 106. Therefore, under a certain condition of reproducibility of the driving voltage, the maximum value of the voltage generated by the sensor electrode 5 correlates with a deformation amount in the center axis direction (designated in figure) of the vibration plate 102 at resonance shown in FIG. 1C. In the present embodiment, the amplitude in the center axis direction of each cell detachment device is measured in advance so that the output corresponding to the amplitude of the vibration plate 102 at resonance, which mainly contributes to the cell detachment, is obtained from the sensor electrode 5, and these amplitudes and the output of the sensor electrode 5 are recorded in the storage section 205, for example. In addition to the method using the storage section 205 or the like, there is a method as a hardware method, of recording the amplitude by fixing it at an arbitrary resistance value with a resistor or the like. In the case of the hardware method, in order to improve workability, a plurality of resistors or the like may be arranged on the substrate in advance so that they can be easily selected by a dip switch or solder or the like.

The determination section 203 records, in advance, in a storage area, a value determined at the time of design, such as, for example, in the case of a wave of natural vibration, two bending shapes are formed in the vicinity of 20 kHz or more. Since the frequency of the vibration mode naturally increases when the wave number of vibration increases, it is preferable to provide the set values of the detection voltage and amplitude of the vibration wave individually. In addition, the determination section 203 compares the recorded value with the voltage and frequency of the alternating current generated by the sensor electrode 5 to grasp the type and intensity of each generated vibration, and the notification control section 204 may notify the user of this by using the monitor 300.

### (EXAMPLE 3)

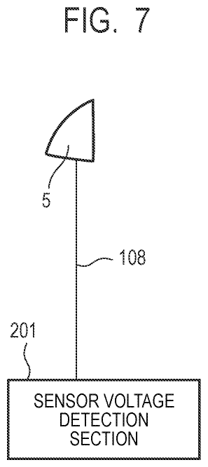

In the cell detachment device used in Examples 1 and 2 described above, the sensor electrode 5 and the sensor voltage detection section 201 are connected by the same ordinary wiring as other wiring. However, the voltage value of the alternating current generated in the sensor electrode 5 is weak and may be affected by the voltage when the alternating current is applied to the driving electrode 7. In the present embodiment, a shield wire having an effect of reducing noise is used for this wiring. As shown in FIG. 7, by connecting the sensor electrode 5 and the sensor voltage detection section 201 with the shield wire 108, an effect of reducing the influence of noise when the alternating current is supplied to the drive electrode 7 is expected.

### (EXAMPLE 4)

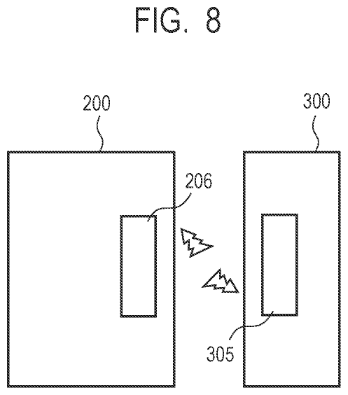

In the above-described embodiment, the control device 200 and the monitor 300 are connected by a wire. However, the control device 200 may be provided with a wireless communication function and connected to the monitor 300 using the wireless communication function. Then, for example, a maintenance worker located in a remote area may be notified via the monitor 300 whether the cell detachment device is normal, abnormal, or has a possibility of disconnection. In this case, the control device 200 may be provided with a transmission/reception section 206, the monitor 300 may be provided with a transmission/reception section 305, and LAN, Wi-Fi, Bluetooth (registered trademark), or the like may be used, as shown in FIG. 8. Further, the functions may be extended by using USB or the like to perform these communications, and a network line provided in advance may be used.

### (Other embodiments)

The present disclosure may also be implemented by supplying a program implementing one or more functions, such as the embodiments described above, to a system or device through a network or storage medium, and by one or more processors in the computer of the system or device reading and executing the program. It may also be implemented by a circuit (For example, ASIC) implementing one or more functions.

As described above, according to the cell detachment device according to the present embodiment, for example, when a disconnection occurs in the device, the maximum voltage amount obtained from the sensor electrode 5 is approximately 0V, even if an alternating current is input to the driving electrode 7, and the possibility of disconnection can be easily sensed. In addition, for example, by obtaining a voltage in previously set predetermined voltage range designating a normal driving state of the cell detachment device, from the alternating current of the sensor electrode 5, it can be easily known that the detachment operation is normally performed. More specifically, the determination section 203 compares the maximum voltage value of the alternating current obtained from the sensor electrode 5 with a range of voltage values set in advance and stored in the storage section 205. For example, when the amplitude applied to the culture vessel 100 in the cell detachment device is lowered, the voltage value obtained from the sensor electrode 5 is lowered. When the determination section 203 determines that the lowered voltage value has fallen below the range of the stored voltage value, the notification control section 204 causes the monitor 300 to inform an abnormality. In addition, since it is assumed that the desired vibration amplitude is given to the culture vessel 100 when the obtained voltage value is within the range of the previously stored voltage value, the notification control section 204 may cause the monitor 300 to inform that the cell detachment device is operating normally. As a result, the user can appropriately know the operation status of the cell detachment device, can easily avoid excessive or insufficient vibration imposition on the cells, and can perform efficient cell detachment processing.

As described in Example 3, when the alternating current obtained from the sensor electrode 5 is weak, the wiring connecting the sensor electrode 5 and the control device 200 may be shielded so as to cope with noise. Also, as described in Example 4, the monitor 300 and the control device 200 may be connected wirelessly or via a network, so that even a maintenance worker or a researcher in a remote area can know whether or not the operation condition of the cell detachment device is normal. Thus, maintenance and inspection of the cell detachment device can be performed effectively.

Here, the sensor electrode 5 is provided in the region where the piezoelectric element 106 vibrates more by the part to be vibrated, so that the state of vibration given to the culture vessel 100 can be grasped more accurately. For this reason, it is desirable that the sensor electrode 5 is arranged so as to be partially on the outer edge side of the cross-sectional center line which is the neutral axis of the ring-shaped piezoelectric element 106. The ripple-shaped vibration wave mode of the vibration generated in the vibrated element consisting of the vibration plate 102 and the piezoelectric element 106 can be changed by selecting the frequency of the stretching vibration of the piezoelectric element 106. At this time, the outer edge of the vibration element is supported by a cushion member to form a substantially free end, so that the ripple-shaped vibration can be formed starting from the cross-sectional center axis of the annular piezoelectric element 106.

Since the outer edge of the piezoelectric element 106 is conventionally used for fixing with a structure in a cell detachment device, the vibration generated here is extremely minute and is not very suitable for obtaining a voltage. In the first embodiment, the outer edge of the vibration element is unrestrained and supported by a cushion member, so that sliding in the radial direction is freely possible and movement in the axial direction is also freely possible. As a result, vibration deformation can be increased at a position outside the cross section neutral axis of the piezoelectric element undergoing deformation of the bimorph and outside the node of the most outer edge ripple. By arranging the sensor electrode 5 in the vicinity thereto, vibration of the piezoelectric element 106 caused by vibration of the vibrated system can be increased and converted into a second alternating current. In addition, by floating in the air through the cushion member, the outer edge becomes free without mechanical support, so that the state of vibration can be detected more correctly without any external hindrance.

In the first embodiment, the sensor electrode 5 outputs the alternating current generated on the piezoelectric element 106 according to the sum of the deformation of the vibration plate 102 and the strain deformation caused by the application of the alternating current to the piezoelectric element 106. Therefore, under a certain condition of reproducibility of the driving voltage, the maximum voltage obtained from the sensor electrode 5 correlates with the amplitude of the center of the vibration plate 102 at resonance. An output corresponding to the amplitude in the center axis direction is obtained from the sensor electrode 5. Therefore, the amplitude in the center axis direction is measured in advance for each cell detachment device, and this is stored in the storage section 205 as a table, which may be used for the determination of the determination section 203. In addition, the voltage corresponding to the amplitude amount may be obtained without using the storage section 205, for example, by a resistor as a hardware method. For example, in a vibration element, the vibration mode is determined at the time of design, for example, in the case of a natural vibration wave, two bending shapes are formed in the vicinity of frequency range of 20 kHz or more. Therefore, the type and intensity of each generated vibration can be easily grasped from the relationship between the voltage obtained from the output of the sensor electrode 5 and the driving frequency. Since the frequency of the vibration mode naturally increases as the wave number increases, the voltage obtained from the output of the sensor electrode 5 and the set value of the amplitude may be provided individually.

In the present embodiment, in order to reduce the inductive voltage in an environment where the driving voltage is disturbed so that the amplitude of the vibration element can be detected more correctly, the lead wire connected to the sensor electrode 5 has a conductor connected to the ground adjacent to it.

As described above, the cell detachment device according to the present embodiment is a cell detachment device that generates first vibration and detach the culture cell from the culture vessel using the first vibration, and comprises a piezoelectric element, a vibrated system, a third electrode, and a unit for detecting the state of the first vibration. The vibration of the piezoelectric element 106 is excited by applying a first alternating current using a first electrode exemplified by the driving electrode 7 and a second electrode exemplified by the ground electrode 6. In the present embodiment, the vibrated system includes, for example, the vibration member (vibration plate 102) to which the piezoelectric element 106 is fixed and the culture vessel 100 mounted on the vibration member, and the second vibration excited by the piezoelectric element 106 propagates. The third electrode exemplified by the sensor electrode 5 is used to output a second alternating current excited by the piezoelectric element due to the first vibration generated in the vibrated system resulting from the propagated first vibration. The unit for detecting the state of first vibration includes, for example, a sensor voltage detection section 201, the determination section 203, and the notification control section 204, and detects the state of first vibration generated in the vibrated system using the output from the sensor electrode 5.

In the present embodiment, the piezoelectric element in which the second alternating current is excited by the first vibration generated in the vibrated system is the piezoelectric element 106 to which the first alternating current is applied. The sensor electrode 5, which is the third electrode, is electrically separated from the drive electrode 7 in the piezoelectric element 106 on the same surface where the drive electrode 7, which is the electrode to which the first alternating current is applied, is provided. In this case, in the present embodiment, the piezoelectric element 106 has a ring shape, and the sensor electrode 5 may be provided so as to partially be on the outer edge side of the center line of the cross section, which is the neutral axis of the ring shape.

The cell detachment device according to the present embodiment may further include the cushion member 110 on which the vibration element exemplified by the vibration plate 102 and the piezoelectric element 106 fixed to the vibration plate are mounted and supported against the mounting surface (119) on which the cell detachment device is mounted. By supporting the vibration element in an unconstrained state by the cushion member 110, the first vibration of the vibrated system can be excited on the piezoelectric element 106 as a larger amplitude. In the present embodiment, the cell detachment device is preferably connected to a display unit, exemplified by the monitor 300, capable of displaying the state of the vibration. In this case, the control device of the cell detachment device may further include a notification control section (notification control section 204) that causes the display unit to display information based on the detected state of vibration and prompts notification to the user.

In addition, the present embodiment may be an embodiment of a vibration element used in the cell detachment device. In this case, the vibration element includes the piezoelectric element 106 having the first and second electrodes (7,6) described above, and a third electrode exemplified by the sensor electrode 5. The present embodiment can also be an embodiment of a control method of a cell detachment apparatus. In this case, the control method includes detecting a second alternating current excited by the piezoelectric element 106 due to first vibration generated in the vibrated system due to the propagated second vibration. The control method also includes determining whether the voltage related to the second alternating current is lower than or equal to a threshold value, and prompting the user to notify the user by displaying on the monitor 300 that the vibration of the vibrated system when the second alternating current is obtained is not appropriate when the voltage is lower than or equal to the threshold value.

As described in Example 4, the cell detachment device according to the present embodiment can be connected to the display unit exemplified by the monitor 300 and the notification control section (204) by wireless communication. Further, the storage section (205) can be further provided which stores the voltage related to the second alternating current correlated with the amplitude amount of the vibration plate 102 output from the sensor electrode 5 and the amplitude amount in the center axis direction of the vibration plate 102 in correspondence with each other. In this case, the determination section (203) may be provided which determines whether the voltage related to the second alternating current is lower than or equal to the threshold value set based on the voltage stored in the storage means (205). In this case, when the determination section determines that the voltage is lower than or equal to the threshold value, the notification control section (204) may be further provided which causes the monitor 300 to display that the vibration of the vibrated system when the second alternating current is obtained is not appropriate and prompts the user to notify the user.

As described above, in the cell detachment device according to the first embodiment, the sensor electrode 5 provided in the piezoelectric element 106 can be used to determine whether or not the culture vessel 100 and the cells are subjected to vibration under a desired condition.

### <SECOND EMBODIMENT>

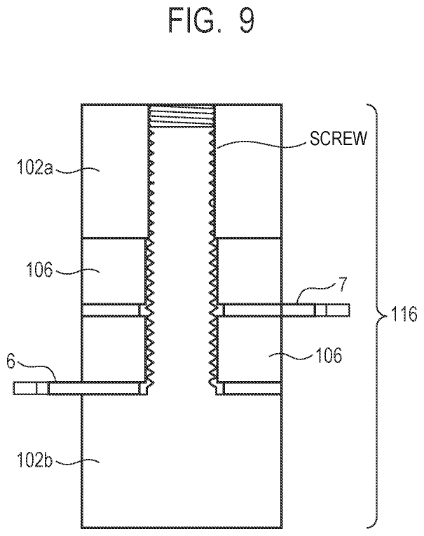

In the first embodiment, a case where the present disclosure is applied to a cell detachment device using as the vibration element, the annular piezoelectric element 106 and the vibration plate 102 to which the piezoelectric element 106 is fixed is described. In contrast, in the second embodiment, the present disclosure is applied to, for example, a Langevin type vibration element or a swinging type vibration element. The second embodiment will now be described with reference to FIG. 9 to FIG. 11C. FIG. 9 shows an axial cross-sectional view of a Langevin type vibration element to which the present disclosure is applied. The Langevin type vibration element 116 uses a first vibration member 102b having a bolt shape and a second vibration member 102a having a nut shape, in place of the vibration plate 102 having a disk shape used in the first embodiment. Between the neck of the first vibration member 102b and the second vibration member 102a, the piezoelectric element 106 to which the ground electrode 6 is attached on one surface thereof, and to which the drive electrode 7 and the sensor electrode attached on the other surface is sandwiched. Since the drive electrode and the sensor electrode are formed in the piezoelectric element 106 as shown in FIGS. 3A and 3B, only the grand electrode 6 and the drive electrode 7 is shown in FIG. 9.

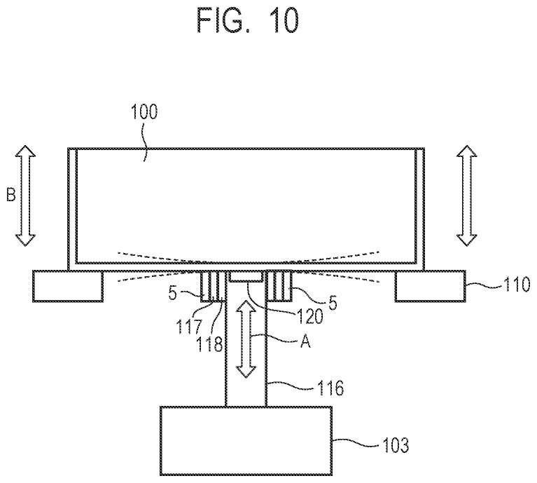

Next, an example in which the above-described vibration element 116 is applied to the cell detachment device will be described with reference to FIG. 10. FIG. 10 is a schematic configuration diagram of the cell detachment device mainly showing the vibration element 116 in the cell detachment device and related configurations. When the vibration element 116 is used, it is not necessary to provide the piezoelectric element 117, the sensor electrode 5, and the ground electrode 118 shown in FIG. 10 and described in detail later. The culture vessel 100 is mounted on the cushion member 110 at the outer periphery of the bottom surface thereof, and the vibration element 116 arranged on the back surface side thereof is fixed to the back surface of the culture vessel 100 by a vacuum chuck 120. In this embodiment, one end of the vibration element 116 is fixed by adsorption to the back surface of the culture vessel 100, and the other end is fixed to a base 103 composed of, for example, a housing of the detachment device. Since the base 103 is composed of a member which does not inhibit vibration in the stretching direction (A direction) of the vibration element 116, vibration in the B direction can be given to the culture vessel 100. Also in this embodiment, by detecting vibration of the piezoelectric element 106 caused by vibration generated in the vibrated system consisting of the vibrating members 102a, 102b, the culture vessel 100, etc. by the sensor electrode 5, the state of vibration generated by the cell detachment device can be known.

In this embodiment, the center of the culture vessel 100 is vibrated. In this case, if the base supporting the vibration element disposed in the center is softened, the outer edge of the vibration element can be vibrated as a substantially free edge as in the first embodiment. In addition, the outer edge of the culture vessel 100 can be vibrated as a substantially free edge as in the first embodiment through an elastic body, for example. With this configuration, a large vibration amplitude is given to the piezoelectric element 106 provided with the sensor electrode 5, and a more accurate vibration state can be detected.

In the case of the present embodiment, in order to more accurately detect the vibration generated in the vibrated system, a piezoelectric element for vibration detection can also be provided in order to more accurately detect expansion and contraction of the vibration element in the axial stretching direction (the vertical direction corresponding to the A direction in the figure) or the radial stretching direction. In this case, the sensor electrode 5 for the piezoelectric element 106 is not formed as described in FIG. 9. Then, for example, as shown in FIG. 10, the ground electrode 118 is fixed to the side surface of the vibration element 116, and a piezoelectric element 117 composed of, for example, a thin film is formed thereon so as to extend in the extending direction of the vibration element 116. The sensor electrode 5 is further formed on the piezoelectric element 117. By converting the stretching vibration of the vibration element 116 into an alternating current through the piezoelectric element 117 and detecting this by the sensor electrode 5 and the ground electrode 118, it is possible to more accurately detect the vibration of the vibrated system. It is also desirable that the piezoelectric element 117 be arranged closer to the culture vessel 100 as shown in FIG. 10 so that the vibration of the vibrated system can be more accurately detected. Here, an electrode arranged on the vibration element 116 side of the piezoelectric element 117 is used as a ground electrode, and an electrode arranged on the opposite side is used as a sensor electrode, but these electrodes may be arranged in reverse.

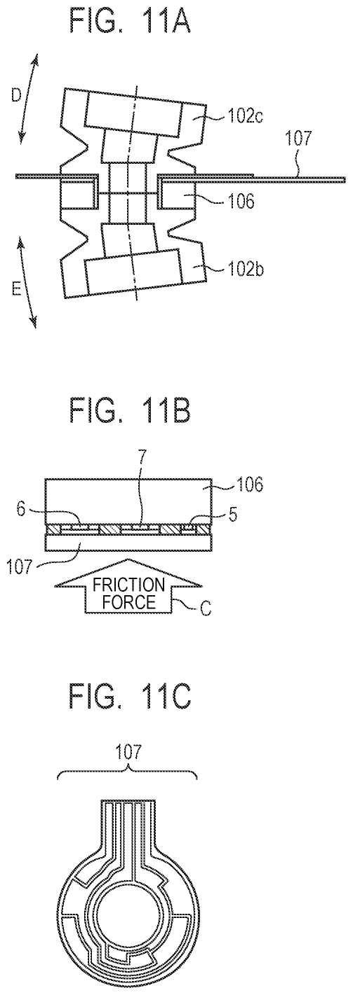

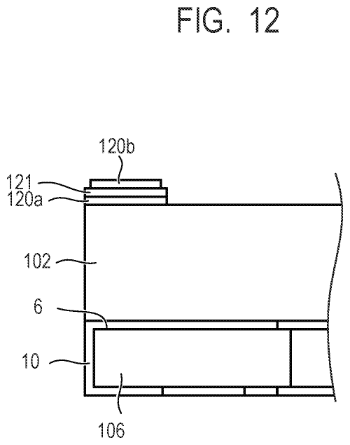

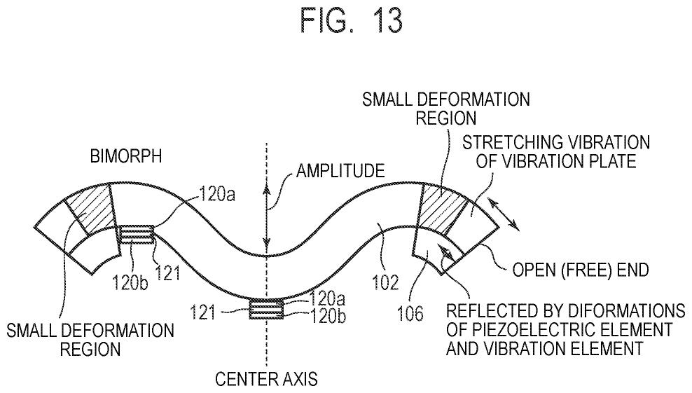

Next, a case where the present disclosure is applied to the cell detachment device using a swinging type vibration element will be described with reference to FIGS. 11A to 11C. FIG. 11A shows an axial section of a swinging type vibration element to which the present disclosure is applied, FIG. 11B shows the relationship between the piezoelectric element 106 and the formation arrangement of each electrode, and FIG. 11C shows a top view of a flexible printed circuit board from which wiring is drawn from each electrode.

In the swinging type vibration element, in place of the ting-shaped vibration plate 102 used in the first embodiment, the substantially annular first vibration member 102c and the second vibration member 102d are used by being fixed in the vertical direction by using a bolt, and the piezoelectric element 106 is sandwiched between the vibrating members. The driving electrode 7 and the sensor electrode 5 are provided on one surface of the piezoelectric element 106, and the ground electrode 6 is formed in the substantially entire area of the other surface so as to be drawn from the same surface to the other surface. A wiring forming surface of a flexible printed circuit board 107 is arranged so as to face each other on this one surface, and the wiring and the electrode are brought into close contact by friction force C when the first vibration member 102c and the second vibration member 102d are fixed, thereby ensuring electrical conduction. By applying an alternating current to the driving electrode 7, the piezoelectric element 106 expands and contracts, and a swinging vibration indicated by an arrow D is generated in the first vibration member 102c, and a swinging vibration indicated by an arrow E is generated in the second vibration member 102d. By applying these vibrations to the culture vessel 100, cell detachment can be promoted. Although the vibration mode of the swinging vibration element is different from that of the Langevin type vibration element 116 described above, such vibration element can be used instead of the Langevin type vibration element 116. Again, by using the alternating current obtained from the sensor electrode 5, it is possible to detect the state of the vibrations applied to the vibrated system. Also, in the present vibration element, it is possible to detect the state of the vibrations applied to the vibrated system by using the same configuration as in the case where the piezoelectric element 117 is used.

As described above, in the cell detachment device according to the second embodiment, the piezoelectric element in which the second alternating current is excited by the vibrations generated in the vibrated system can be provided as the piezoelectric element 117 different from the piezoelectric element 106 in which the first alternating current is applied. The second piezoelectric element exemplified by the piezoelectric element 117 is fixed to the vibration element consisting of the first piezoelectric element exemplified by the piezoelectric element 106 in which the first alternating current is applied and the vibration members 102a and 102b. The third electrode (sensor electrode 5) is provided so as to output the second alternating current excited by the second piezoelectric element (117). In this case, the piezoelectric element 106 has a ring shape, and the second piezoelectric element (117) is provided so as to output the second alternating current excited in the piezoelectric element 106 by the axial stretching vibration or the radial stretching vibration of the ring shape by the vibration generated in the vibrated system.

In addition, the vibration element consisting of the piezoelectric element 106 and the vibration plate 102 can come into contact with the bottom surface in an area smaller than the bottom surface of the culture vessel 100. In this case, the culture vessel 100 can be supported against the mounting surface (119) through the cushion member 110.

As described above, in the cell peeling apparatus according to the second embodiment, it is possible to know whether or not the culture vessel 100 and the cells are subjected to vibration under a desired condition by using the sensor electrode 5 provided on the piezoelectric element 106 or the piezoelectric element 117.

### <THIRD EMBODIMENT>

In the first and second embodiments described above, the case where the piezoelectric element 106 is provided with the sensor electrode 5 for detecting the alternating current generated in the piezoelectric element 106 due to vibration of the vibrated system is described. In the second embodiment, the vibration element 116 is provided with the piezoelectric element 117 for generating the alternating current due to vibration of the vibrated system regardless of the arrangement of the piezoelectric element 106. Further, the piezoelectric element 117 is provided with the sensor electrode 5 to detect the alternating current of the piezoelectric element 117. In the present embodiment, in the vibration element used in the first embodiment, a piezoelectric element causing the alternating current to occur due to the vibration of the vibrated system is provided on the vibration plate 102, and a sensor electrode is provided on the piezoelectric element. By obtaining the alternating current of the piezoelectric element, the state of vibration applied to the vibrated system to is known.

FIG. 12 shows a cross section of an essential part of a vibration element consisting of the piezoelectric element 106 and the vibration plate 102 provided with a piezoelectric element for vibration detection of the vibrated system according to the present embodiment. In figure the ground electrode 6 formed on the entire upper surface of the piezoelectric element 106 through a folding part 10 on the lower surface is shown. On the upper surface of the vibration plate 102, a thin-film ground electrode 120a, a piezoelectric element 121, and a sensor electrode 120b are formed in this order from the surface of the vibration plate 102. An alternating current also occurs in the piezoelectric element 121 due to vibration of the vibrated system. By obtaining this alternating current using the sensor electrode 120b, the state of vibration applied to the vibrated system can be detected. In the first embodiment, since the culture vessel 100 is placed approximately around the center of the vibration plate 102, it is preferable that these piezoelectric elements 121 and the like are arranged on the outer periphery. In the present embodiment, the ground electrode 120a is provided corresponding to the piezoelectric element 121, but the ground electrode 6 for driving the piezoelectric element 106 can also be used as the ground electrode for detecting the alternating current. In the present embodiment, the electrode arranged on the vibration plate 102 side of the piezoelectric element 121 is used as the ground electrode, and the electrode arranged on the opposite side is used as the sensor electrode, but these electrodes may be arranged in reverse.

### (MODIFIED EMBODIMENT)

For example, in an environment where the driving voltage applied to the piezoelectric element 106 is disturbed, it is necessary to take measures to make the amplitude amount more correctly detectable. For this purpose, in the present modification, another piezoelectric element for detecting the alternating current caused by the vibration of the vibrated system is fixed to a rear surface side of the vibration plate 102 on the portion of the vibration plate 102 that is directed to an antinode of the vibration of the deformation of the vibration plate 102, especially on the antinode of the vibration wave at the outermost edge. The reason why the piezoelectric element is fixed to the rear surface side is that the culture vessel 100 is mounted on a front surface side, and the surface of the vibration plate 102 is basically not provided with irregularities. In addition, a flammable liquid-like vibration transmitter may be used, and the upper surface is preferable for safety

The piezoelectric element 121 for detecting the alternating current is provided with the ground electrode 120a and the sensor electrode 120b, as in the third embodiment described above. Note that the piezoelectric element 121 is preferably lightweight and small in a range that does not affect the vibration of the vibrated system, and may be provided by, for example, baking an electrode, a piezoelectric element, or the like as a thin film. The sensor electrode 120b or the like may be formed by plating or the like. Alternatively, a flexible substrate or the like may be used.

In the present embodiment, in order to reduce the inductive voltage in an environment in which the driving voltage is disturbed so that the amplitude amount of the vibration element can be detected more correctly, a lead wire connected to the sensor electrode 5 has a conductor connected to the ground adjacent to it. In addition, in order to enable to detect the amplitude amount of the vibration element more correctly in an environment in which the driving voltage is disturbed, the piezoelectric element different from the electrode for applying the alternating current to the ring-shaped piezoelectric element 106 is provided on a portion of the antinode of the ripple at the outermost edge of the vibration plate 102. Thus, the influence of the driving vibration of the piezoelectric element 106 can be reduced, and the vibration component of the vibration plate 102 can be detected.

As described above, in the cell detachment device according to the third embodiment, the piezoelectric element 121 in which the second alternating current is excited by the vibration generated in the vibrated system is different from the first piezoelectric element exemplified by the piezoelectric element 106 in which the first alternating current is applied. The second piezoelectric element exemplified by the piezoelectric element 121 is fixed to the vibration member (102). In this case, the third electrode exemplified by the sensor electrode 120b is provided so as to output a second alternating current excited by the second piezoelectric element (121). The second piezoelectric element may also be provided, as exemplified by the piezoelectric element 117, corresponding to the position where the antinode of the amplitude of vibration generated at the outermost edge of the vibration member (102) occurs.

As described above, the cell detachment device according to the third embodiment uses the sensor electrode (5, 120b) provided on the second piezoelectric element (117) provided on the vibration plate 102. By using these sensor electrodes, it is possible to determine whether or not the culture vessel 100 and the cells are subjected to vibration under desired conditions.

Although the present disclosure has been described with reference to embodiments and examples, the present disclosure is not limited to the above-described embodiments and examples. The present disclosure also includes inventions that have been modified to the extent not inconsistent with the purpose of the present disclosure and inventions that are equivalent to the present disclosure. In addition, the foregoing embodiments and embodiments may be combined as appropriate to the extent not inconsistent with the purpose of the present invention.

According to one embodiment of the present disclosure, it is possible to determine whether the culture vessel and the cells are provided with vibrations of the desired condition in the cell detachment device.

### <OTHER EMBODIMENT>

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™M), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-192053, filed Nov. 30, 2022, which is hereby incorporated by reference herein in its entirety.

What is claimed is:

1. A cell detachment device generating a first vibration and detaching a culture cell from a culture vessel by using the first vibration, comprising:
a piezoelectric element in which a second vibration is excited by applying a first alternating current using a first electrode and a second electrode;
a vibrated system in which the second vibration propagates, including a vibration member to which the piezoelectric element is fixed and on which the culture vessel mounted;
a third electrode used configured to output a second alternating current excited on the piezoelectric element by the first vibration generated in the vibrated system caused by the second vibration; and
a detection unit configured to detect a state of the first vibration generated in the vibrated system by using the second alternating current output from the third electrode.

2. The cell detachment device, according to claim 1, wherein the first alternating current is applied to the piezoelectric element on which the second alternating current is excited by the first vibrations generated in the vibrated system, and
the third electrode is provided separately from one of the first electrode and the second electrode, to which the first alternating current is applied, on the same surface of the piezoelectric element as the surface of the piezoelectric element on which the one of the first electrode and the second electrode, to which the first alternating current is applied.

3. The cell detachment device according to claim 2, wherein the piezoelectric element has a ring shape, and
the third electrode is provided so as to be partially on an outer edge side of the cross sectional center line, which is the neutral axis of the ring shape.

4. The cell detachment device according to claim 1, wherein the piezoelectric element comprises:
a first piezoelectric element in which the second vibration is excited by applying the first alternating current using the first electrode and the second electrode; and
a second piezoelectric element in which the second alternating current is excited by the first vibration generated in the vibrated system, and which is different from the first piezoelectric element, and is fixed to a vibrator element comprising the first piezoelectric element and the vibrating member, and
the third electrode is provided so as to output the second alternating current excited by the second piezoelectric element.

5. The cell detachment device according to claim 4, wherein the first piezoelectric element has a ring shape, and
the second piezoelectric element is provided to output the second alternating current excited by an axial stretching vibration generated in the first piezoelectric element by the first vibration generated in the vibrated system, or by a radial stretching vibration of radial direction the ring shape.

6. The cell detachment device, according to claim 1, wherein the piezoelectric element comprises:
a first piezoelectric element in which the second vibration is excited by applying the first alternating current using the first electrode and the second electrode; and
a second piezoelectric element in which the second alternating current is excited by the first vibration generated in the vibrated system, and which is different from the first piezoelectric element, and is fixed to the vibrator element, and
the third electrode is provided so as to output the second alternating current excited by the second piezoelectric element.

7. The cell detachment device according to claim 6, wherein the second piezoelectric element is provided corresponding to a position where an antinode of an amplitude of the second vibration generated at an outermost edge of the vibration member generates.

8. The cell detachment device according to claim 1, further comprising a cushion member on which the vibration member and the piezoelectric element fixed to the vibration member are mounted, and which is supported against a surface on which the cell detachment device is mounted.

9. The cell detachment device according to claim 4, wherein the vibration element comprising the first piezoelectric element and the vibration member can abut against a bottom surface of the culture vessel in an area smaller than the bottom surface of the culture vessel, and the culture vessel is supported against a surface on which the cell detachment device is mounted via a cushion member.

10. The cell detachment device according to claim 1, further comprising:
a display unit capable of displaying the state of the first vibration; and
a notification control unit configured to cause the display unit to display information based on the detected state of the first vibration on the display means so as to prompt notification to a user.

11. The cell detachment device according to claim 10, wherein the display unit and the notification control unit are connected by a wireless communication.

12. The cell detachment device according to claim 1, further comprising a storage unit configured to store a voltage related to the second alternating current correlated with an amplitude amount of the vibration member, output from the third electrode, by making the voltage in correspondence with the amplitude amount of the vibration member in a center axis direction.

13. The cell detachment device according to claim 12, further comprising: a determination unit configured to determine whether the voltage related to the second alternating current is less than or equal to a threshold value set based on the voltage stored in the storage unit; and
a notification control unit configured to cause a display unit to display information that the first vibration of the vibrated system when the second alternating current is obtained is not appropriate, so as to prompt notification to a user, in a case where the voltage is less than or equal to the threshold value.

14. A vibration element used in a cell detachment device configured to generate a first vibration and to use the first vibration to detach a culture cell from a culture vessel, comprising:
a piezoelectric element in which a second vibration is excited by applying a first alternating current using a first electrode and a second electrode; and
a third electrode used for outputting a second alternating current excited by vibration generated on a vibrated system including a vibration member to which the piezoelectric element is fixed and on which the culture vessel mounted, due to propagation of the second vibration.

15. A method for controlling a cell detachment device that generates a first vibration and uses the first vibration to detach a culture cell from a culture vessel, the cell detachment device comprising: a piezoelectric element in which a second vibration is excited by applying a first alternating current using a first electrode and a second electrode; and a vibrated system in which the second vibration propagates, including a vibration member to which the piezoelectric element is fixed and on which the culture vessel mounted, the method comprising the steps of:
detecting a second alternating current excited by the piezoelectric element by the first vibration generated in the vibrated system caused by the second vibration;
determining whether a voltage related to the second alternating current is lower than or equal to a threshold value; and
causing a display unit to display information that the first vibration of the vibrated system at a time of obtaining the second alternating current is not appropriate in a case where the voltage is less than or equal to the threshold value.

16. A non-transitory tangible medium having recorded thereon a program for causing a computer to perform steps of the method according to claim 15.
